(19) United States
(12) Patent Application Publication
(10) Pub. No.: US 2016/0263221 A1
(43) Pub. Date: Sep. 15, 2016

(54) MULTI-RESPONSIVE TARGETING DRUG DELIVERY SYSTEMS FOR CONTROLLED-RELEASE PHARMACEUTICAL FORMULATION

(71) Applicants: George KORDAS, NEA PENTELI ATTIKIS (GR); Eleni EFTHIMIADOU, ATHENS (GR)

(72) Inventors: George KORDAS, NEA PENTELI ATTIKIS (GR); Eleni EFTHIMIADOU, ATHENS (GR)

(21) Appl. No.: 15/035,855

(22) PCT Filed: Nov. 21, 2014
(86) PCT No.: PCT/EP2014/003113

§ 371 Date: May 11, 2016

(30) Foreign Application Priority Data
Nov. 22, 2013 (GR) 20130100654

(51) Int. Cl. A61K 41/00; A61K 47/48; A61K 31/704

(52) U.S. Cl. CPC A61K 41/0028; A61K 31/704; A61K 47/48869; A61K 47/48176

(57) ABSTRACT

The present invention proposes the development of technology in the domain of targeted therapy and local drug delivery to the affected area of the tumor. The invention relates to surface modification nano-/micro-containers (spheres of organic and inorganic polymers), which are sensitive to acidic environment, elevated temperature (T˜41-43° C.), redox potential as well as the application of external applied magnetic field. These nano-/micro-containers are made suitable to be able to carry drugs, such as antibiotics, anti-cancer, cytostatic and antimicrobials and their release will be carried to the patient tissue due to the prevailing conditions. The composition of nano-/miocro-containers based on organic and inorganic polymers which exhibit sensitivity to the aforementioned conditions. The adjustment is carried out through the surface with aminosilanes or amino acids as ligands or small molecules such maleamide molecules which can undergo nucleophilic addition and further binding of targeting molecule.

### TECHNICAL FIELD OF THE INVENTION

The present invention belongs to the field of pharmaceutical formulation, in particular it related to drug delivery systems that are capable of releasing therapeutic agents at desired sites of actions.

### BACKGROUND OF THE INVENTION

Many traditional medicines, especially chemotherapy medicines, inevitably accompanied devastating side-effects mainly because the medicines not only can destroy the malfunctioned cells but also impose harmful effects on the normal cells in the body. In order to reduce the toxic side-effects and enhance the therapeutic effectiveness of the medicines, various controlled-release drug delivery systems have been proposed and tested.

In general, controlled-release drug delivery systems are stimuli-responsive materials that are capable of preventing interaction between the drugs and the healthy cells during transport and releasing the drugs at desired sites of action. The strategy takes advantage of the different characteristics between healthy cells and cancer cells. Compared to healthy cell, cancer cells exhibit acidic pH because of hypoxia and production of lactic acid in the Krebs cycle, elevated temperature due to the rapid proliferation and higher redox environment.

Targeting drug delivery systems are typically categorized by two main mechanisms, namely passive targeting and active targeting. Passive targeting takes advantage of the relative higher permeability of the tumor tissue as compared to the normal tissue. Size of the delivery vehicle is preferably in the range of about 50-300 nm for passive targeting. Active targeting is usually achieved by directing the delivery vehicles to a targeting moiety, thereby allowing preferential accumulation of the drug at the tumor tissue. Size of the delivery vehicle is generally greater than 1 micron for active targeting.

A wide variety of controlled-release drug delivery systems are under development in research laboratories. Some are now in clinical use, or making their way through trials, including liposomes, micelles and polymeric nano-/micro-particles. These new formulation options offer a few advantages including reducing drug toxicity, reducing the risk of cells developing resistance to the therapy and increasing the availability of expensive therapeutic agents.

CA2821109 discloses a method for preparing cross-linked polymeric micelles as delivery vehicles for chemotherapeutic agents. Doxorubicin is covalently grafted to the hydrophilic shell of the micelles and Curcumin is entrapped within the hydrophobic core of the micelles. Curcumin is a potent inhibitor of the three major drug efflux transport proteins (which is believed to be one of the most important reason for drug resistance in chemotherapy), which helps Doxorubicin to accumulate within the site of action in cancer cells without being effluxed.

WO2011/113616 disclosed pH-sensitive micelles formed by amphiphilic block-copolymer suitable for encapsulating Gadolinium (Gd³⁺) complex as “smart” contrast agents in magnetic resonance imaging (MRI). These micelles are capable of carrying the Gd-complex in the inactive state under normal body pH condition and releasing the Gd-complex and activate its imaging property under mildly acidic condition.

WO2008/115641 disclosed an effective vaccine delivery system which allows the vaccine composition to be orally administered. This is accomplished by encapsulating any vaccine antigen within the nanoparticles together with the activators of the desired immune activity. The delivery system protects the vaccine composition from hydrolysis and degradation in low pH environments and endosome-disrupting agents.

US2006/222594 disclosed a targeting magnetic nanosphere preparation which is effective in diagnosis and treating tumors, the preparation being prepared by encapsulating magnetic iron oxide particles are in a size of tens to hundreds of nanometers using biodegradable polymer having high affinity for biological tissues, allowing penetration into deep areas of biological tissues and detection by MRI, and surface modifying the resulted capsules with an antibody specific to a specific tumor.

US2010/168044 disclosed super-paramagnetic nanoparticles comprising a core which is magnetic field responsive, the surface of said core are conjugated with a therapeutic agent (e.g. Doxorubicin) and said conjugated therapeutic agent and said core are encapsulated in a layer of stimuli-responsive polymer. Said stimuli-responsive polymer may also be conjugated with folic acid for tumor targeting.

WO98/33478 disclosed a pharmaceutical composition suitable for encapsulating biomolecules such RNA- and DNA-based active agents or oligonucleotides in nanoparticles.

CN101920024 disclosed a brain targeted liposome preparation encapsulating ^{<99m>}Tc tumor imaging medicament, wherein a bradykinin analogue RMP-7 is spread on the surface of the liposome as a brain-targeted guiding molecule.

WO2008/066507 disclosed a targeting delivery system for biomolecules such DNA, peptides and antigen. The delivery system protects the encapsulated DNA from GI environment till they reach the target site where, upon degradation, they would release the DNA molecules for gene therapy.

WO94/02106 describes microcapsules in an appropriate form for loading and functionalization of a broad spectrum of drugs and diagnostic agents for targeted delivery in the desired area.

WO92/16212 disclosed compositions and methods for enhancing the delivery in vivo and in vitro of therapeutic agents and other substances to cells, by specifically targeting such agents to the cells (e.g. macrophages) via the lectins thereon.

GR1007882 disclosed multi-stimuli responsive nano- and/or micro-containers without surface modification. The containers disclosed in this patent exhibit no targeting ability and no response to hyperthermia conditions.

While prior art controlled-release drug delivery systems are known and are generally suitable for their limited purposes, they possess certain inherent deficiencies that reduce their overall utility in drug delivery.

### BRIEF DESCRIPTION OF FIGURES

FIG. 1: A drawing illustrating the multi-responsive targeting nano-/micro-containers of the present invention, wherein 1 indicates the Fe₃O₄ nanoparticles, 2 indicates the receptor or specific molecules, 3 indicates the fluorescent probe, 4 indicates the Gd-DOTA contrast agent which is optional, 5 indicates the drugs, and 6 indicates the multi-sensitive polymeric layer.

FIG. 2: Schematic representation of the maleamide linker.

FIG. 3: Reaction scheme of Fe₃O₄ magnetite nanoparticles deposit and subsequent modification with folic acid moiety.

FIG. 4: TEM images of the micro-containers before and after the magnetic nanoparticle modification.

FIG. 5: Demonstration of the targeting ability.

FIG. 6: Reduction of tumor volume after I.V. injection of Doxorubicin loaded multi-responsive targeting nano-containers of the present invention.

### SUMMARY OF THE INVENTION

The present invention relates to a technology for targeted therapy and localized drug delivery to an affected tissue within the human body. In particular, the present invention provides containers (organic and/or inorganic polymer containers, preferably spheres) that are sensitive to acidic environment, elevated temperature (T˜41-43° C.), redox potential, as well as the application of an external stimulus. These containers are made suitable for carrying drugs, such as antibodies, anti-cancer, cytostatic and antimicrobials, and releasing the drugs in the affected tissues by responding to the local environment changes caused when healthy cells turn into diseased cells. Diseased cells, such as cancerous cells, show changes in temperature, pH and redox potential all due to the higher metabolic rate.

Additionally the invention relates to such containers that are surface modified such that one or more targeting molecule is installed onto the surface of these containers in order that the drug-loaded containers are more likely to be targeted and retained in the affected diseased cell.

Additionally the invention relates to such surface modified containers that are only capable of releasing the drug upon the cell being subjected to an external stimulus—such as a magnetic field, radio frequency, ultrasound, photon and laser. Such external stimuli can be directed to diseased cells locally within the body and provided the container has the appropriate material the container will respond by heating up further and releasing the active pharmaceutical substance. This allows even higher doses of containers to the administered since accidental release in other parts of the body is minimized and release in the diseased cell is maximized.

Accordingly, the present invention provides containers having at least one pharmaceutical substance and adapted to release the at least one pharmaceutical substance inside a diseased cell, characterized in that:
a) the containers are made from materials that respond to at least one of the following stimuli that is found to be different inside the diseased cell compared with a non-diseased cell, pH, temperature and re-dox environment;
b) the containers have targeting molecules that are capable of recognizing receptors that are overexpressed on the diseased cell;

wherein upon stimulation by one or more of the above mentioned stimuli, the containers release the pharmaceutical ingredient inside the diseased cell.

The present invention further provides a process for the preparation of the above mentioned hollow polymeric containers, comprising the following steps of:
(a) preparing cores having the desired size;
(b) preparing polymeric shells by wrapping the cores with materials that respond to at least one of the following stimuli that is found to be different inside the diseased cell compared with a non-diseased cell, pH, temperature and re-dox environment;
(c) removing the cores and providing containers;
(d) attaching targeting molecules on the surface of the containers obtained by step (c);
(e) optionally depositing metallic nanoparticles on the surface of the containers obtained by steps (c) or (d); and
(f) loading active pharmaceutical substances onto the hollow polymeric containers obtained by step (e).

Further aspects of the invention are use of containers as described herein in medical therapy, specifically in the treatment of cancer or any other disease mentioned herein.

Another aspect of the invention is the use of the containers as described herein for targeting controlled-release delivery of an active pharmaceutical compound. Preferred actives are any of those mentioned herein.

A further aspect of the invention is a method of treating a disease (as mentioned herein) in a human in need thereof which method comprises administering a therapeutically effective amount of a container as described herein containing an active pharmaceutical compound (as mentioned herein) for that disease (the disease mentioned herein) and, optionally, where a contrast agent is in the container, performing a scan (preferably a MRI scan) to ensure the containers are in the diseased cell and then, optionally, applying an external stimuli as described herein where the containers have a nanoparticle metal.

### DETAILED DESCRIPTION OF THE INVENTION

Definitions and abbreviations:

As used herein, the term “APTES” means (3-aminopropyl)triethoxysilane.

As used herein, the term “boc” means tert-butyloxycarbonyl protecting group.

As used herein, the term “cbz” means Carboxybenzyl protecting group.

As used herein, the term “CDDP” means cisplatin, cisplatinum, or cis-diamminedichloroplatinum(II).

As used herein, the term “cys” means cysteine.

As used herein, the term “DIC” means N, N-diisopropyl carbodiimide.

As used herein, the term “DLS” means dynamic light scattering.

As used herein, the term “DMAEMA” means N, N-dimethylaminoethyl methacrylate

As used herein, the term “DMF” means dimethyl formamide.

As used herein, the term “DMSO” means dimethyl sulfoxide.

As used herein, the term “DNR” means daunorubicin.

As used herein, the term “DOX” means doxorubicin.

As used herein, the term “DS” means N,N′-[disulfanediyl-bis(ethane-2,1-diyl)]bis(2-methyl acrylamide)

As used herein, the term “DVB” means divinyl benzene.

As used herein, the term “EDAX” means energy dispersive X-ray analysis.

As used herein, the term “EG” means ethyleneglycol.

As used herein, the term “EGF” means epidermal growth factor.

As used herein, the term “EtOH” means ethanol.

As used herein, the term “FITC” means fluorescein isothiocyanate.

As used herein, the term “FT-IR” means Fourier transform infrared spectroscopy.

As used herein, the term “GFLG” means Gly-Phe-Leu-Gly tetra-peptide.

As used herein, the term “GnRH” means Gonadotropin-releasing hormone.

As used herein, the term “GPCRs” means G protein-coupled receptors.

As used herein, the term “HETM” means hexamethyl tetramine.

As used herein, the term “HPMA” means N-(2-hydroxypropyl)methacrylamide.

As used herein, the term “IOZ” means 2-isopropyl-2-oxazoline.

As used herein, the term “KPS” means potassium persulphate.

As used herein, the term “lys” means lysine.

As used herein, the term “MMA” means methyl methacrylate.

As used herein, the term “MRI” means magnetic resonance imaging.

As used herein, the term “NIPAAm” means N, N-isopropylacrylamide.

As used herein, the term “OH” means hydroxyl or hydroxyl.

As used herein, the term “PBS” means phosphate buffered saline.

As used herein, the term “PET” means Positron Emission Tomography.

As used herein, the term “pyroGlu” means pyroglutamic acid.

As used herein, the term “QS” means catalytic amount.

As used herein, the term “RGD” means Arg-Gly-Asp structure.

As used herein, the term “rpm” means revolutions per minute.

As used herein, the term “SCID” means severe combined immunodeficiency.

As used herein, the term “SEM” means scanning electron microscope.

As used herein, the term “TEA” means triethylamine.

As used herein, the term “TEM” means Transmission electron microscopy.

As used herein, the term “VCL” means N-vinylcaprolactam.

As used herein, the term “VEGFR” means Vascular Endothelial Growth Factor Receptor.

As used herein, the term “VME” means vinyl methyl ether.

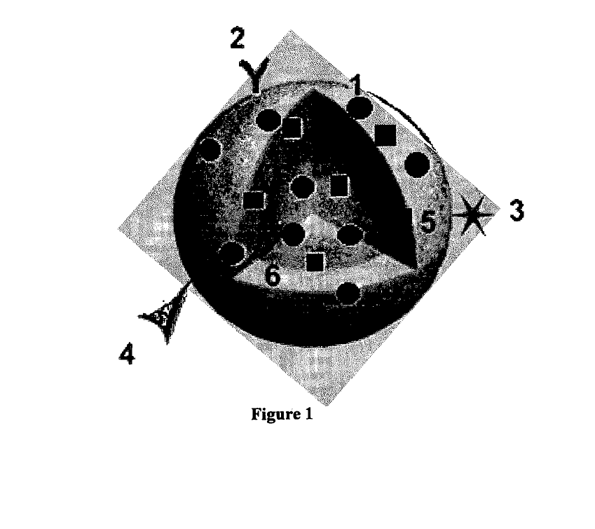

A drawing illustrating the multi-responsive targeting nano-/micro-containers of the present invention is displayed in FIG. 1. The nano-/micro-containers fabrication is based on a three-step procedure. The first step involves the synthesis of the cores, which are usually spherical particles having controlled sizes. The second step involves the synthesis of the shells, which are usually cross-linked polymeric coatings wrap around the cores prepared in the first step. At the end of the second step, the cores are removed to create hollow cavities inside the shells. The third step involves the installation of targeting molecules in the outer surface of the shell. These targeting molecules recognize their sites of action through attraction towards a specific receptor on the cancerous cells.

The nano-/micro-containers of the present invention are vary in size from 100 nm to 10 microns as determined by conventional particle size measuring techniques. SEM and TEM can be used to determine the particle size in the solid state. DLS can be used to investigate the behavior of the nano-/micor-containers in solution.

SEM and TEM images were obtained on an FEI Inspect microscope operating at 25 kV and a FEI CM20 microscope operating at 200 kV, equipped with a Gatan GIF200 Energy Filter utilized for EF-TEM elemental mapping respectively.

Hydrodynamic diameter (D_{H}) and colloidal stability of the containers of the present invention were studied by using the dynamic light scattering technique. DLS measurements were performed on a Malvern Instruments Zetasizer Nano Series instrument, with a multipurpose titrator. Distilled water and 5 mM PBS were used as the dispersant medium. The concentration of the containers used in the DLS study is preferably 25 mg/L.

It is observed that the minimum hydrodynamic size is slightly bigger than the diameter measured in solid state using SEM and TEM. This is due to the surface roughness of the containers which are different from the perfect sphere model that DLS used for size calculations. In all, the DLS results agree closely with the values obtained by SEM and TEM.

The sizes of the containers depend on the sizes of the cores prepared in the first step. When the polymerization is carried out in heterogeneous medium, particles having regular shapes and controllable size can be obtained, for example emulsion polymerization, dispersion polymerization, suspension polymerization, precipitation polymerization, etc.

The cores can be made of organic or inorganic materials. Useful inorganic materials for the cores are silicon oxide (SiO₂) particles prepared by sol-gel methods. Useful organic materials for the cores are polymeric particles. Preferred in the present invention are methacrylate type polymeric particles. Water-soluble copolymer of poly(MMA-co-HPMA) are most preferred.

The shells are selected from polymers which exhibit the desired properties that responsive to different pH, temperature and redox environment. These polymers exhibit a response to one stimulus or a combination of stimuli and can be synthetic or natural polymers. Preferably, these polymers are cross-linked.

For pH-responsive shell, monomers can be selected from methacrylic acid, acrylic acid, didsoproyl amino ethyl methacrylate, maleic anhydride, DMAEMA, N, diethyl acrylate, or mixture thereof. For thermo-responsive shell, monomers can be selected from NIPAAm, DMAEMA, VCL, IOZ, VME, HPMA and mixture thereof. For re-dox sensitive shell, monomers can be selected from bis-methacryloyl cysteamine, diallyl disulfide and mixture thereof. The release of the drug-load is based on electrostatic, hydrophobic, and hydrogen bonding interactions.

Once the shells are formed, the cores can be removed by treatment with a good solvent for the core materials but a non-solvent for the shell materials. Treatment can be performed during the synthesis process or afterward with appropriate solvent, such as chloroform. Treatment with water during the synthesis process is preferred.

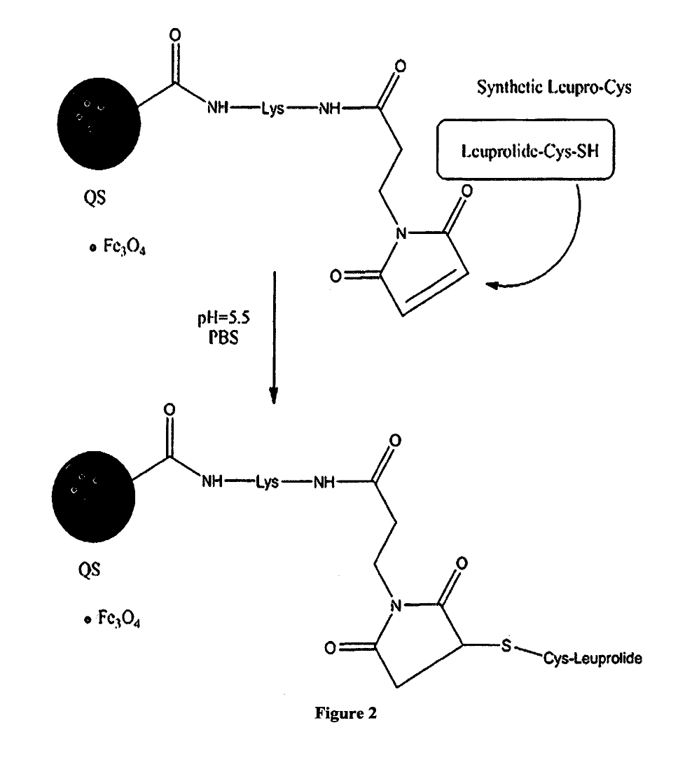

Targeting molecules are installed on the surfaces of the nano-/micro-containers through linkers. Useful linkers contain at least two functional groups. One of them is reactive to the functional groups on the outer surface of the shell and another one of them can react with the functional groups on the targeting molecules; hence connecting the two together. Suitable linkers include GFLG peptide, Cbz-Lys(boc)-, and maleamide linkers. Preferred in the present invention is the maleamide linker. FIG. 2 shows the scheme of a maleamide linker connecting the nano-container and Leuprolide-Cys (a peptide as targeting GnRH agent).

Suitable targeting molecules are selected from a group consisting of small molecule receptors, such as folic acid, enzyme receptors, such as tyrosine kinases, EGF, transmembrane receptors, such as GPCRs, antigens, other overexpressed receptors and contrast agents. Particularly suitable targeting molecule/receptor pairs include: folic acid—folate receptor; quinazolines and other VEGFR analogues—Tyrosine Kinase; hyalouronic acid—CD-44 receptor; RGD—integrins a1b1. In addition, antigens and peptides can also be used to promote targeting attachment. For example, Rituximab is a monoclonal antibody that targets the cell surface antigen CD20;

Trastuzumab is a monoclonal antibody that targets the cell surface receptor of HER-2/neu(erb-2); Cetuximab is an antigen against the epidermal growth factor receptor and has been proved for treatment of refractory colon cancer. Furthermore, surface receptor(s) also exist for lung cancer cells and are peptide ligands specific. Such targeting molecule and receptor pairs are well known in the art. Optionally contrast agents are added to the container so that the doctor may see that the containers have been absorbed into the damaged cells before applying any external stimuli. Examples of suitable contrast agenst are those used in MRI (such as gadolimium based agent including gadoterate, gadodiamide, gadobenate, gadopentetate,gadoteridol, gadofosveset,gadoversetamide, gadoxetate, and gadobutrol and other magnetic agents such as ironplatinum and manganese).

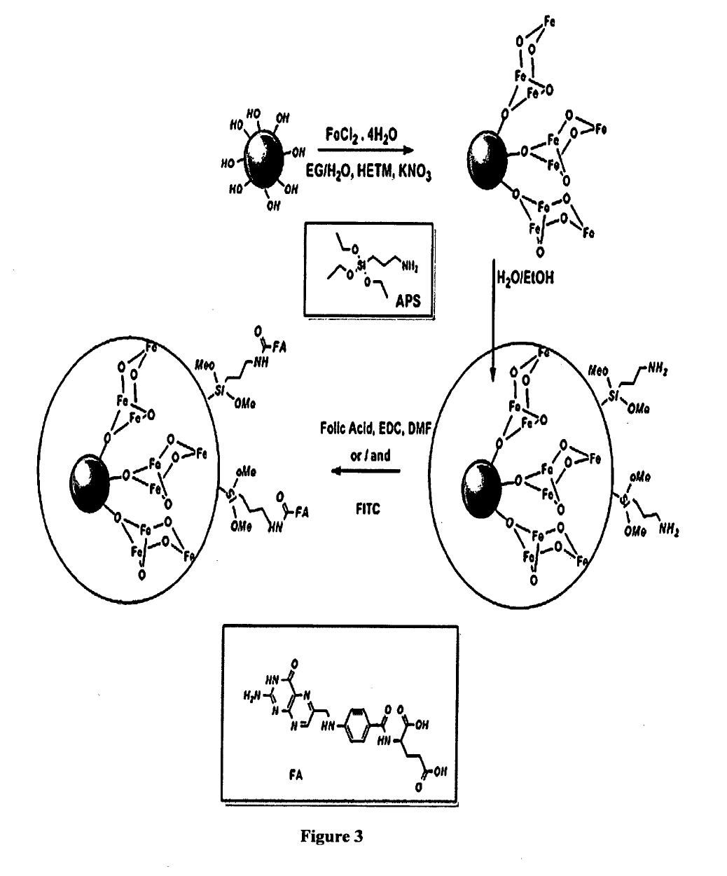

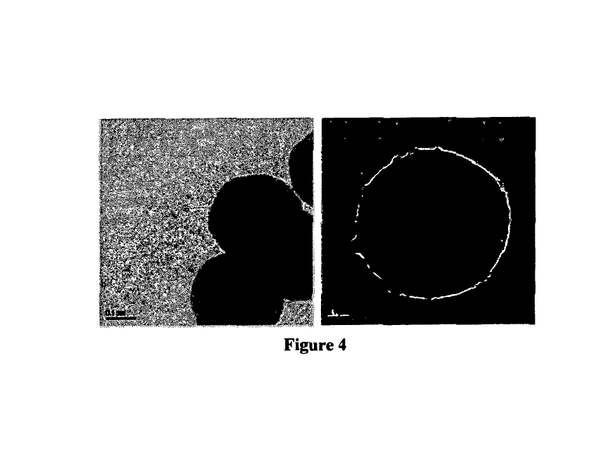

The nano-/micro-containers of the present invention may be further modified with magnetic and/or gold nanoparticles so that these materials can respond to external stimuli, such as magnetic field, light, sonication. Magnetite (Fe₃O₄) nanoparticles are particularly preferred. FIG. 3 displays the reaction scheme of the deposit of magnetite nanoparticle and the subsequent modification with folic acid. In addition, the iron nanoparticle can also be used in hyperthermia applications. FIG. 4 shows TEM images of the micro-containers before and after magnetic nanoparticle modification.

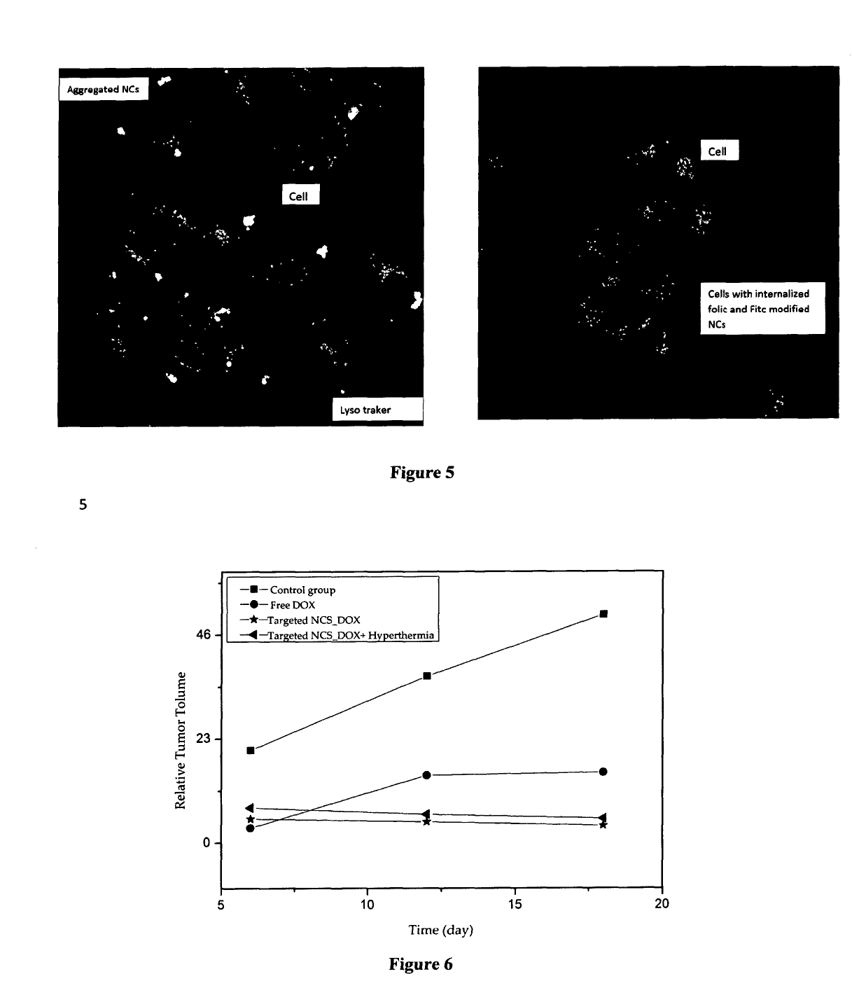

The targeting ability is demonstrated in FIG. 5. Both in vivo and in vitro experiments are demonstrated here. Two parallel experiments where the nano-containers are installed with and without folic acid, as targeting molecules, are conducted. The nano-containers without folic acid agglomerate outside the cells (FIG. 5, left). The nano-containers attached with folic acid are located on the cell surface (FIG. 5, right). The experiments are repeated in vivo using SCID mice where the nano-containers are grafted with technetium to allow PET measurement in order to count the number and location of the nano-containers. It is observed that the nano-containers are passively localized in lungs.

The multi-responsive targeting nano-/micro-containers of the present invention may be used to carry a wide variety of active pharmaceutical substances, including antibiotics, peptides, anticancer substances, natural products, proteins, genetic material, cells, microorganisms, antimicrobials. Preferred drugs include anthracyclines, such as doxorubicin, daunorubicin, epirubicin, idarubicin, valrubicin and mitoxantrone, Quinolones, such as levofloxacin and ciprofoxacin, taxanes such as docetaxel, paclitaxel, taxotere and paclitaxel, Curcumine, cyclosporine, Gemcitabine, platinum based drugs such as cisplatin and oxaliplatin.

A typical procedure for the loading of the active substance, for example Doxorubicin, in the containers of the present invention is as follows. Equal amounts of the containers of the present invention and the drug were treated under neutral conditions (pH=7.4, PBS buffer solution) and the mixture was gently stirred for three days in the dark at room temperature. After that, the mixture was centrifuged three times (10000 rpm×5 min) and the isolated products were washed three times by PBS.

The loading of the drugs onto the containers can be attributed to the electrostatic interactions between functional groups on the drug molecules and the carboxylic acid groups on the containers. The unloaded drug was estimated in the supernatant according to a DOX calibration curve in PBS. The Loading capacity (LC) and encapsulation efficiency (EE) of the containers and the exact mass of the loaded drug (as one embodiment: 486±2.1 μg/mg of polymer) inside the containers can also be calculated.

In the release study a calculated amount of drug loaded containers was suspended in two different pH conditions, pH=4.5 and pH=7.4, and/or in combination of glutathione. After that, 0.5 mL of the suspension was removed by centrifugation and the released drug was calculated in the supernatant.

The inventors determine the therapeutic efficacy of the multi-responsive targeting nano-containers of the present invention using HeLa tumor bearing SCID mice and monitor the volume of cancer as a function of time. FIG. 6 shows the results of these experiments. The squares indicate the growth of cancer as a function of time in the SCID mice treated with DOX-loaded nano-containers without folic acid targeting. The dots indicate treatment with Doxorubicin. It can be observed in the figure that, in both of these cases, the volume of the cancer increases with time. On the other hand, when treated with DOX-loaded nano-containers with folic acid targeting (stars), a 20% volume reduction is observed in twenty days. The same experiment was repeated using hyperthermia (triangles) and the application of hyperthermia gives improved therapeutic outcome (better reduction of cancer volume as a function of time).

### EXAMPLES

The described invention is further enhanced by the introduction of following schemes and examples.

### Example 1

(1.1) Synthesis of Poly(MMA-co-HPMA) as Core

MMA (1 ml), HPMA (30 g) and 25 ml water are mixed in a flask and the mass is warmed up to 75° C. under nitrogen atmosphere for about 1 hour. KPS (20 mg) is added to the mixture and the reaction mass is kept until the polymerization is completed (usually an overnight reaction is required). After the reaction mass is cooled to room temperature, the mixture is centrifuged for 5 minutes at 12000 rpm for three times. The morphological and structural characterization of the copolymer cores can be measured using SEM, FT-IR and DLS. The averaged particle size of the cores is 180±20 nm in diameter as determined by SEM.

| Reagents | Amounts | |
|---|---|---|
| Methyl methacrylate | 1 | ml |
| N-hydroxy propyl methacrylate | 30 | mg |
| Potassium persulfate | 20 | mg |
| water | 10 | g |

(1.2) Synthesis of the Poly(MMA-co-HPMA-co-DS-co-DVB) as Shells

The poly(MMA-co-HPMA) cores are dispersed in 25m1 mixture of deionized water and EtOH (H₂O/EtO H=20/5) with the aid of ultra-sonication. MMA (300.8 mg, 320 μL, 3.004 mmol), HPMA (60 mg, 0.419 mmol), DS (30 mg, 0.104 mmol) and DVB (137.1, 150 1.053 mmol) were added and the dispersion and the mass is stirred for 30 min under nitrogen purge. KPS (0.02 g, 2% w/w of the monomers) was added into the dispersion aiming at initiating the polymerization. The polymerization was maintained for about 12 hours at 70° C. The resultant poly(MMA-co-HPMA) cores in poly(MMA-co-HPMA-co-DS-co-DVB) shells were purified by dispersion in water/ethanol mixture to remove the unwanted oligomers and unreacted monomers. The products were washed by three cycles of centrifugation with deionized water in order to remove the poly(MMA-co-HPMA) cores. Then the solids are dried in a vacuum oven. The molar percentages of the monomers in the shells are: MMA 65%, HPMA 9%, DS 2.2% and DVB 22%. According to this ratio, the resulted microspheres are about 300 nm in diameter.

| Reagents | Amounts | |
|---|---|---|
| PMMA-co-HPMA cores | 150 | mg |
| Methyl methacrylate | 3.004 | mmol |
| N-hydroxy propyl methacrylate | 0.419 | mmol |
| DS | 0.104 | mmol |
| DVB | 1.053 | mmol |
| Potassium persulfate | 20 | mg |

| | |
|---|---|
| Water:EtOH | 23:2 (total volume 25 ml) |

(1.3) Synthesis of Poly(MMA-co-HPMA-co-DS-co-DVB) Hollow Microspheres

The isolated poly(MMA-co-HPMA-co-DS-co-DVB) hollow microspheres were dispersed in a solution consisting of ethylene glycol (EG) and water in volume ratio of EG/H₂O equals to 65/35. The mixture is maintained for about 1 hour under nitrogen and then 100 mg hexamethyl tetramine and 80 mg Iron (III) chloride tetrahydrate (FeCl₃.4H₂O) were added thereto. After 20 minutes of stirring, 80 mg potassium nitrate (KNO₃) was introduced in the reaction solution and the mass was heated at 80° C. for 4 hours.

A key factor for the formation of magnetite nanoparticles lies in that the pH of the solution should be at 9.0±0.5.

| Reagents | Amounts | |
|---|---|---|
| Hollow nanospheres | 100 | mg |
| Ferrous chloride tetrahydrate | 80 | mg |
| Potassium nitrate | 80 | mg |
| Hexamethyl tetramine | 100 | mg |
| EG/H₂O | 65/35 | ml |

After the reaction is completed, the reaction mass is cooled to room temperature and the magnetic functionalized hollow microspheres were purified with water washing and centrifuged. The iron percentage is about 5% according to SEM-EDAX measurement and this is the optimal percentage for hyperthermia application.

(1.4) Modification of the Surface With Inorganic Polymer

Nano-/micro-containers are dispersed in an ethanol solution for about 30 minutes and then added APTES. The mixture was stirred and then added drop-wise to a solution of ammonia. The mixture was allowed to stir 24 hours. Then the mixture was centrifuged and washed several times with water and ethanol and then was dried.

| Reagents | Amounts | |
|---|---|---|
| Nano-containers | 10 | mg |
| APTES | 300 | μl |
| Water/Ethanol | 20/80 | |
| NH₃ | 20 | μl |

(1.5) Surface Binding of Folic Acid

In a 50 ml flask, the nano-containers obtained by (1a) are dispersed in a solution of TEA/DMF and the mass is stirred for 30 minutes. In another flask, folic acid is introduced to a solution of DMSO and then DIC is added. The mixture is left for 30 minutes with stirring and transferred to the first flask. The reaction was allowed 24 hours to complete and subsequently the mixture is centrifuged and washed with DMF. The solids are dried and characterized. The determination of the concentration of folic acid was made by measuring the unbounded folic acid.

| Reagents | Amounts | |
|---|---|---|
| Modified Nano-containers | 10 | mg |
| Folic acid | 5 | mg |
| DMSO | 1 | ml |
| DMF | 500 | μl |
| DIC | 100 | μl |

(1.6) Surface Binding of Fluorescein

Nano-containers which have been modified with folic acid are dispersed in a solution of TEA/DMF. Then FITC is added and the solution is allowed to react for 5 hours.

The mixture was centrifuged and the wash solution is collected.

| Reagents | Amounts | |
|---|---|---|
| Nano-comtainers modified with folic acid | 10 | mg |
| FITC | 5 | mg |
| DMSO/DMF | 2 | ml |
| TEA | Cat. Amt. | |
| DIC | 50 | μl |

(1.7) Gold Nanoparticles Modification

The isolated hollow microspheres (11.23 mg) were dispersed in 5 ml phosphate buffer. The mixture is maintained for 1 h and then 3.78 mg of HAuCl₄ was added and the mixture and was stirred at 50° C. After 1 h stirring the reaction was cooled down at room temperature and the magnetic functionalized hollow microspheres were purified with water.

| Reagents | Amounts | |
|---|---|---|
| Modified nano-containers | 11.23 | mg |
| HAuCl₄ | 4 | mg |
| Phosphate buffer saline | 5 | ml |

### Example 2

### Leuprolide Binding

(2.1) Modification of the Surface With Lysine

Nano-containers are dispersed in a flask containing a solution of TEA/DMF and the mixture is stirred. Lysine, having the functional groups protected, is added in another flask having the same solvent. DIC is added and allowed to stir. After 30 minutes the mixture was added to the original and was allowed to stir. After 24 hours the mixture was centrifuged and the collected product is washed and dried. The product was characterized by scanning microscopy and infrared spectroscopy. The product is treated with 20% pyridine in DMF for 2 hours.

| Reagents | Amounts | |
|---|---|---|
| Modified nano-containers with | 10 | mg |
| NH-Lys(NH₂)—COOH | | |
| Maleamide | 60 | mg |
| DIC | 200 | μl |
| TEA | Cat. Amt. | |
| DMF | 2 | ml |

(2.2) Surface Modification With Maleamide Linker

The lysine-modified particles are modified and on the amine with a molecule containing maleimide moiety according to the carbodiimide chemistry, which has been described above.

| Reagents | Amounts | |
|---|---|---|
| Nano-containers | 10 | mg |
| Fmoc-NH-Lys(NH₂)—COOH | 30 | mg |
| DIC | 5 | mg |
| TEA | Cat. Amt. | |
| DMF | 2 | ml |

(2.3) Modification of the Surface With Cysteine-Grafted Leuprolide and Fluorescein

The maleimide-modified nano-containers are dispersed in a phosphate buffer (pH=5.5) and then the cysteine-grafted Leuprolide is added. It is allowed to stir and then centrifuged, washed with purified water and then dried. Then the binding process of fluorescein isothiocyanate is as described in (1.6).

| Reagents | Amounts | |
|---|---|---|
| Modified nano-containers with | 4 | mg |
| Maleamide-NH-Lys(NH₂)—COOH | | |
| Cysteine-grafted Leuprolide | 0.04 | mg |
| Phosphate buffer pH = 5.5 | 2 | ml |

1. Containers having at least one pharmaceutical substance and adapted to release the at least one pharmaceutical substance inside a diseased cell, characterized in that:
a) the containers are made from materials that respond to at least one of the following stimuli that is found to be different inside the diseased cell compared with a non-diseased cell, pH, temperature and re-dox environment;
b) the containers have targeting molecules that are capable of recognizing receptors that are overexpressed on the diseased cell;
wherein upon stimulation by one or more of the above mentioned stimuli, the containers release the pharmaceutical ingredient inside the diseased cell.

2. Containers as claimed in claim 1 wherein the container is made from a material that responds to a change in temperature.

3. Containers as claimed in claim 2 wherein the containers have metallic nanoparticles.

4. Containers as claimed in claim 3 wherein the metallic nanoparticles respond to an external stimulus by creating heat.

5. Containers according to claim 4 wherein the external stimuli used comprise one of magnetic field, radio frequency, ultrasound, photon, laser, or any combination thereof.

6. Containers according to claim 1 having a size in the range of from 100 nm to 10 microns in diameter.

7. Containers according to claim 1, wherein the targeting molecules comprise one of folic acid, VEGFR analogues, Hyaluronic acid or RGD.

8. Containers according to claim 7, wherein the targeting molecule is folic acid.

9. Containers according to claim 1, wherein the targeting molecules are connected to the container through a linker.

10. Containers according to claim 9, wherein the linker is a maleamide linker.

11. Containers according to claim 1, wherein the metallic nanoparticles are magnetite (Fe₃O₄) or gold nanoparticles.

12. Containers according to claim 3, wherein the metallic nanoparticles are gold nanoparticles.

13. Containers according to claim 1, wherein the active pharmaceutical substances comprise one of an anthracycline, a taxane, curcumine, doxorubicin, gemcitabine or cisplatin.

14. Containers according to claim 10, wherein the active pharmaceutical substance is doxorubicin.

15. A process for the preparation of containers according to claim 1, comprising the following steps of:
(a) preparing cores having the desired size;
(b) preparing polymeric shells by wrapping the cores with materials that respond to at least one of the following stimuli that is found to be different inside the diseased cell compared with a non-diseased cell, pH, temperature and re-dox environment;
(c) removing the cores and providing containers;
(d) attaching targeting molecules on the surface of the containers obtained by step (c);
(e) optionally depositing metallic nanoparticles on the surface of the containers obtained by steps (c) or (d); and
(f) loading active pharmaceutical substances onto the hollow polymeric containers obtained by step (e).

16. A process according to claim 15, wherein the cores are made of organic materials or inorganic materials.

17. A process according to claim 16, wherein the cores are made of organic polymers, which can be homopolymers or copolymers.

18. A process according to claim 17, wherein the cores are made of copolymer of poly(MMA-co-HPMA).

19. A process according to claim 18, wherein the polymeric shells are cross-linked copolymers.

20. A process according to claim 19, wherein the polymeric shells are cross-linked copolymers of poly(MMA-co-HPMA-co-DS-co-DVB).

21. A process according to claim 20, wherein the targeting molecules comprise one of folic acid, VEGFR analogues, Hyalouronic acid, or RGD.

22. A process according to claim 21, wherein the targeting molecule is folic acid.

23. A process according to claim 22, wherein the targeting molecules are connected to the containers through a linker.

24. A process according to claim 23, wherein the linker is a maleamide linker.

25. A process according to claim 24, wherein the metallic nanoparticles are magnetite (Fe₃O₄) nanoparticles.

26. A process according to claim 25, wherein the metallic nanoparticles are gold nanoparticles.

27. A process according to claim 12, wherein the active pharmaceutical substances comprise one of anthracyclines, taxanes, curcumine, Gemcitabine or Cisplatin.

28. A process according to claim 12, wherein the active pharmaceutical substance is Doxorubicin.

29. (canceled)

30. (canceled)

31. (canceled)

32. (canceled)
